(19) United States
(12) Patent Application Publication
(10) Pub. No.: US 2021/0109087 A1
(43) Pub. Date: Apr. 15, 2021

(54) APPARATUS FOR ESTABLISHING THE EFFECT OF ACTIVE INGREDIENTS ON NEMATODES AND OTHER ORGANISMS IN AQUEOUS TESTS

(71) Applicant: Bayer Aktiengesellschaft, Leverkusen (DE)

(72) Inventors: Franco FOIS, Monheim (DE); Michael HARNAU, Leichlingen (DE); Klaus OCHMANN, Leverkusen (DE)

(21) Appl. No.: 16/464,414

(22) PCT Filed: Nov. 22, 2017
(86) PCT No.: PCT/EP2017/080017

§ 371 Date: May 28, 2019

(30) Foreign Application Priority Data
Nov. 30, 2016 (EP) 16201303.1

(51) Int. Cl. G01N 33/50; G01N 21/84; H04N 5/225

(52) U.S. Cl. CPC G01N 33/5085; G01N 21/84; G01N 2333/4353; G01N 2201/0631; G01N 2201/0621; H04N 5/2256

(57) ABSTRACT

The invention relates to an apparatus (1) for establishing the effect of active ingredients on nematodes and other organisms in aqueous tests, comprising a holder (40) for receiving a cell culture plate (50) with wells (57), in which the nematodes with the active ingredients can be filled, wherein the cell culture plate (50) has a bottom side (51), a top side (52) and a plurality of side faces, which extend between the bottom side (51) and top side (52), a camera (20) that serves to record images of preferably the bottom side (51) of the cell culture plate (50), and an illumination device (30) for illuminating the cell culture plate (50), wherein the illumination device (30) comprises a plurality of light sources. According to the invention, in the use position of the cell culture plate (50), provision is made for the light of a first light source (31) to enter the cell culture plate (50) from the outside through a first side face (53), the light of a second light source (32) to enter the cell culture plate (50) from the outside through a second side face (54), the light of a third light source (33) to enter the cell culture plate (50) from the outside through a third side face (55) and the light of a fourth light source (34) to enter the cell culture plate (50) from the outside through a fourth side face (56). Moreover, the invention relates to a method for adjusting the individual light sources.

The invention relates to an apparatus for establishing the effect of active ingredients on nematodes and other organisms in aqueous tests. Moreover, the invention relates to a method for adjusting an illumination device of the apparatus.

Many species of nematodes (roundworms) represent agricultural pests, since they can severely impair plant metabolism as a result of their penetration into the root systems. Various chemical substances, the so-called nematicides, have already been developed against an attack by nematodes. However, there is a great demand to identify further active ingredients which can control nematodes effectively.

WO 2016/116291 A1 has disclosed an apparatus for establishing the effect of active ingredients on nematodes, by means of which a multiplicity of active ingredients can be tested within a short period of time. Here, the individual wells of a cell culture plate, which are filled with nematodes and different active ingredients, are examined simultaneously. Here, the cell culture plate held by a holder of the apparatus has a bottom side, a top side and four side faces that extend between the bottom side and the top side of the cell culture plate. The apparatus comprises a camera that serves to record images of the bottom side of the cell culture plate, and hence of all wells at the same time. An illumination device of the apparatus has two opposing light sources, which illuminate the cell culture plate.

It was found that the application of the apparatus from WO 2016/116291 A1 reaches its limits when examining very small roundworms such as dirofilaria. Although the resolution of the apparatus can be increased, in principle, by selecting a correspondingly higher resolving camera, it was found that inhomogeneities in the illumination of the cell culture plate can no longer be corrected to a satisfactory degree by way of software in the case of a relatively high resolution. Consequently, a reliable and robust establishment of the effect of the active ingredients is no longer possible in the case of very small organisms.

Therefore, the invention is based on the object of providing an apparatus for establishing the effect of active ingredients on nematodes and other organisms, by means of which a reliable establishment of the effect of the active ingredients is possible, even in the case of very small organisms such as dirofilaria.

The object underlying the invention is achieved by using an apparatus according to claim 1. Exemplary embodiments of the invention can be gathered from the dependent claims relating to claim 1.

The apparatus according to the invention is distinguished by virtue of, in each case, the light of a first light source entering the cell culture plate from the outside through a first side face, the light of a second light source entering the cell culture plate from the outside through a second side face, the light of a third light source entering the cell culture plate from the outside through a third side face and the light of a fourth light source entering the cell culture plate from the outside through a fourth side face. Consequently, light enters the interior of the cell culture plate from all side faces. In principle, a uniform illumination of the cell culture plate within the meaning of the invention is possible as a result of this measure. Consequently, the effect of active ingredients even on very small organisms can be examined in cell culture plates in the case of a correspondingly powerful camera. Consequently, it is possible to obtain robust results in the case of a resolution of 30 μm or better. In one exemplary embodiment, the resolution is better than 20 μm, for example 15 μm.

An intensity of the light can be set individually for the first light source. This also applies analogously to the second light source, the third light source and the fourth light source. By way of example, it is consequently possible to set the intensity of the light of the first light source independently of the intensity of the light of the second light source or any other light source. Ultimately, the four light sources are thus able to emit light into the cell culture plate with different light intensities in each case.

The first light source can be arranged laterally next to the first side face, and so, in the use position of the cell culture plate (the cell culture plate extends in a horizontal plane in this case), a light beam of the first light source reaches in substantially horizontal fashion into the cell culture plate through the first side face. Consequently, possible deflection mirrors or other optical deflections can be dispensed with.

The explanations in relation to the first light source should also apply analogously to the other light sources. Thus, for example, the second light source, too, can be arranged laterally next to the second side face. The features that relate to the first light source and that are described below can also be implemented in a single one of the remaining light sources, in some of the remaining light sources or else in all of the remaining light sources.

The invention assumes a rectangular cell culture plate, which has a rectangular basic form with two parallel side faces in each case. By way of example, the ratio of long edges (long side faces) to short edges (short side faces) can be 3 to 2. The wells are usually arranged in a plurality of rows that are arranged parallel to one another. By way of example, a cell culture plate can have 8 rows with 12 wells in each case. Typically, pursuant to the ANSI standard, rectangular microplates with 6, 12, 24, 96 or 384 wells are used.

The invention is based on the discovery that the good illumination of all wells of the cell culture plate, which is required when examining very small organisms, can be implemented by means of an illumination device if light enters into the cell culture plate from all side faces. In the case of a hexagonal cell culture plate, the corresponding basic form of which, however, is currently uncommon, all six side faces would have to be irradiated with light according to the invention. In the case of a triangular cell culture plate, the use of only three light sources would be sufficient.

Typically, the side faces extend between top side and bottom side of the cell culture plate, which has light-transmissive walls.

The first light source typically extends over the entire first side face, as a result of which a uniform irradiation of the first side face is possible.

In one exemplary embodiment, a vertical height in relation to the cell culture plate, an inclination angle about an own longitudinal axis and/or a horizontal distance from the first side face can be set individually for the first light source by way of means for adjusting the illumination device.

The illumination of the cell culture plate can be optimized as a result of the flexible and individually adjustable setting of each light source. Here, seen spatially, each light source is adjustable in three dimensions (height, horizontal distance, inclination angle). Additionally, a fourth dimension can be set, specifically the light intensity. It was found that precise individual setting of the light source on each of the side faces is of great importance for the quality of the measurements, even in the case of a symmetric cell culture plate with a rectangular basic form.

Preferably, each light source is connected to a controller. Particularly preferably, each light source is assembled on a holder, which is connected to the controller or a separate controller.

A vertical height of the light beam emerging from the first light source can be 2 to 6 mm, preferably 3 to 5 mm. In the case of the lateral arrangement of the first light source, this light beam without an inclination angle enters horizontally into the cell culture plate through the first side face, i.e. between the top side and bottom side.

Preferably, an aperture angle of the light beam is very small so that, for example, a cover film that covers the individual wells of the cell culture plate is not illuminated and consequently unable to cause interference effects. However, larger values for the aperture angle can also be tolerated if, for example, only a small vertical portion of the cell culture plate is imaged in focus by the camera and so the plane of the cell culture plate in which the cover film is situated is no longer in focus. In this embodiment, all incoming radiation is kept in such a way that the cell culture plate is only illuminated from the bottom up to the depth of field defined by the lens. The cover or protective film is preferably not illuminated in order to avoid light reflections.

Preferably, the controller is configured to automatically carry out a method for adjusting the illumination device.

The first light source can have a number of light-emitting diodes (LEDs) that are arranged next to one another. In order to produce a uniform light over an effective length of the first light source, the first light source can have a Fresnel lens arrangement. As a result of this Fresnel lens arrangement, the first light source consequently emits a light beam whose intensity is constant when seen over the longitudinal extent of the light source.

The holder for the cell culture plate (also referred to as an MTP holder) can have a plurality of receiving corners, a first receiving corner serving to receive one end of the first side face and a second receiving corner serving to receive an opposite end of the first side face. Here, a distance between the first receiving corner and the second receiving corner is preferably greater than a length of a row of wells, which extends along the first side face. The light of the first light source can consequently enter the cell culture plate unhindered over the entire length of the row of wells. Preferably, the holder is embodied in such a way that no other obstacles are arranged between the first light source and the first side face of the cell culture plate in order to facilitate an unhindered and non-shadowed entry of the light of the first light source into the cell culture plate.

The camera can have a telecentric lens for a precise measurement (e.g. made by Sill Optics) in order to exclude or at least largely exclude a perspective aberration of the image at the edges of the cell culture plate.

Typically, use is made of a digital camera with an image sensor. Preferably, the bottom of the cell culture plate is adjusted in relation to the image sensor surface (also referred to as chip tilt) and the cell culture plate is rotated relative to the image sensor in such a way that the cell culture plate is adjusted parallel to the image sensor (preferably surface and sides) with the greatest possible accuracy. For adjustment purposes, the MTP holder is typically adjustable in precise fashion in all directions, for example by means of clamping with a defined strength.

As a result of applying a telecentric lens in combination with an adjustable cell culture plate assembled on an MTP holder, it was possible to achieve a distortion of <+/−2%, i.e. a flat image recording without software correction. Moreover, a small depth of field can be set by way of the aperture of the telecentric lens, as a result of which interference effects, caused by the aforementioned cover films for the wells, for example, can be reduced.

The apparatus according to the invention facilitates the image analysis of complete plate surfaces and is suitable for cell culture plates with up to 384 wells with small organisms such as dirofilaria (resolution limit approximately 15 μm), for example. Clearly, whole-area images of cell culture plates containing organisms or particles suspended in solutions can be recorded, in general, with the aid of the apparatus according to the invention and can be processed by means of image analysis.

During the operation of the apparatus, the camera records a plurality of digital images of the entire bottom side of, for example, a cell culture plate comprising 96 wells at time intervals of 1 to 5 seconds, for example. By way of an evaluation of these images on the basis of a pixel analysis, for each individual well a certain characteristic for the mean speed with which the dirofilaria situated in this well move, for example, can be established. Here, as a matter of principle, the mean speed in a well with an effective active ingredient is lower than in a well without an active ingredient. Consequently, the effectiveness of an active ingredient can be estimated on the basis of the mean speed. In this context, reference is made to WO 2016/116291 A1, which, in detail, describes a corresponding method for establishing the effect of active ingredients, it being possible to operate the apparatus according to the invention on the basis of said method.

In the case of a wholly uniformly illuminated cell culture plate and in the case of wells that are filled to the same extent in each case (for example without an active ingredient in each case), the same mean speed for the movement of the dirofilaria should theoretically arise for each well if the necessary present biological variance is excluded. By contrast, the results deviate significantly from one another despite the same fill if the illumination of the wells is too poor. Therefore, a statement about the individual active ingredients would not be robust in the case of a cell culture plate with wells filled with different active ingredients that is illuminated in this way. However, the illumination device of the apparatus according to the invention renders it possible to keep the influence of the differences in the illumination, which cannot be entirely precluded in practice, so small that a robust statement about the effect of active ingredients is possible, even in the case of very small dirofilaria (i.e. in the case of a correspondingly high resolution).

Moreover, the object consisted in providing a method for adjusting the illumination device of the apparatus described above in its various embodiments. This object is achieved by the feature combination according to claim 12. Exemplary embodiments of the method according to the invention can be gathered from the dependent claims relating to claim 12.

For each of the light sources, the method according to the invention provides for the relative height in relation to the cell culture plate, the inclination angle, the horizontal distance to the adjoining side face and the light intensity to be set individually in order to obtain an illumination of the individual wells of the cell culture plate that is as uniform as possible.

In a systematic process, individual parameters (height, distance, inclination angle, light intensity) can be set iteratively for each light source and, using these set parameters, it is possible to carry out an above-described establishment of the mean speed for each equally filled well in each case. This process can be repeated as often as desired. The less the established results for the mean speed deviate from one another in the process, the smaller the influence of the non-uniform illumination of the cell culture plate on the trial results. Finally, if the results only deviate from one another with an order of magnitude that can be traced back to biological variance, the method of the adjustment can be completed.

The method for adjusting the illumination device can provide for the camera to be used to create an image of a monochrome sheet which is situated in a plane above a cell culture plate (for example the sheet can be arranged on a non-filled cell culture plate situated in the holder with the interposition of a further empty cell culture plate) and for the adjustment to be implemented or refined on the basis of an evaluation of the image. By way of example, a histogram of the light intensity can be produced from the image and the adjustment can be implemented on the basis of this histogram. Uniform illumination of the sheet is provided if the associated histogram in each case has the same light intensity for all sub-regions (these can also be the individual pixels of the digital image), which would mean that the histogram consists of merely one bar with this one light intensity.

In one exemplary embodiment, the illumination device is adjusted, firstly, by means of the results of a cell culture plate with equally filled wells and, additionally, by means of the above-described sheet. The (first) adjustment on the basis of the equally filled cell culture plate and the (post) adjustment by the sheet can be implemented multiple times in succession and/or in the reverse order.

Moreover, the light intensity of the individual light sources can be reduced at the end of an adjustment, in each case by the same magnitude or in proportional fashion such that a predetermined value emerges for the mean light intensity of the image of the monochrome sheet of paper (e.g. a mean value of 128 if the light intensity can assume values from 0 to 256). Then, during the operation of the apparatus according to the invention, each well of the cell culture plate is assigned a corresponding region on the sheet of paper. The values of the light intensity for the pixels of the well, established by the camera, can then be normalized on the basis of the light intensity of the corresponding region on the sheet of paper. By way of example, if a rather brighter region of the sheet of paper has been established for a well, the values for the light intensity established for this well are reduced by the normalization.

The invention should be explained in more detail on the basis of an exemplary embodiment illustrated in the figures. In the figures:

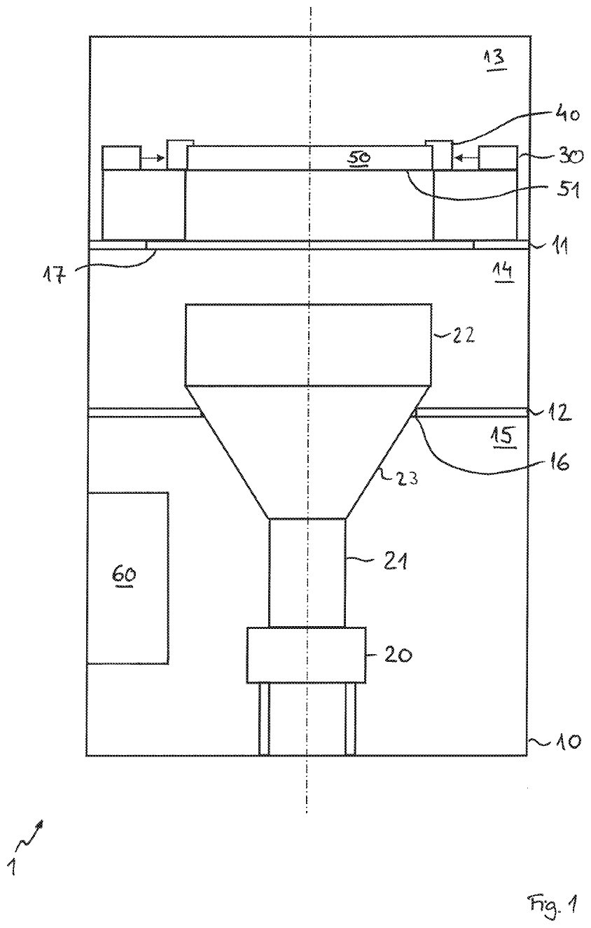

FIG. 1 schematically shows the structure of an apparatus according to the invention;

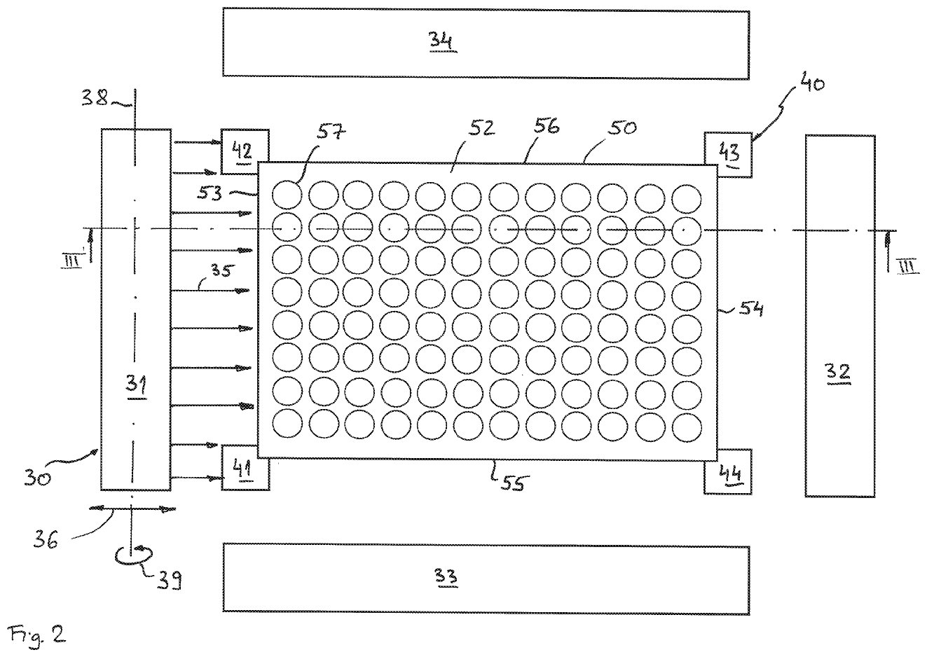

FIG. 2 schematically shows a cell culture plate and an illumination device with four light sources; and

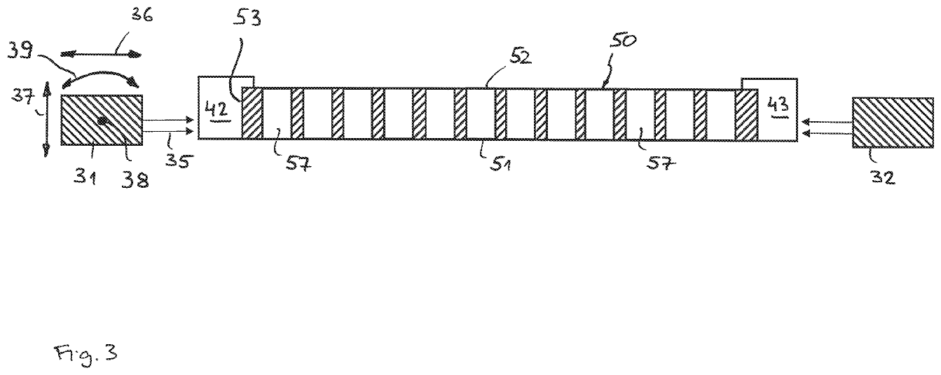

FIG. 3 shows a section along the line III-III in FIG. 2.

FIG. 1 schematically shows an apparatus for establishing the effect of active ingredients on dirofilaria and other organisms in aqueous tests. The apparatus, which is denoted by 1 in the totality thereof, comprises a housing 10 with a top separating plate 11 and a middle separating plate 12. The separating plates 11, 12 divide an interior surrounded by the housing 10 into a top region 13, a middle region 14 and a bottom region 15. A camera 20 is assembled in the bottom region 15. The camera 20 has a telecentric lens 21, the end 22 of which facing away from the camera 20 protrudes into the middle region 14 of the housing 10. The middle separating plate 12 has a circular opening 16, through which the lens 21 passes. Here, a conical-frustum-shaped portion 23 of the lens 21 is supported on the circular edge of the opening 16.

An illumination device 30 and a holder 40 for a cell culture plate 50 are housed in the top region 13 of the housing 10. The illumination device 30, the holder 40 and the cell culture plate 50 will still be described in more detail below on the basis of FIGS. 2 and 3.

In addition to the camera 20, the bottom region 15 of the housing 10 also holds a controller 60 for the camera 20 and the illumination device 30. Here, the controller 60 can consist of separate control units for the camera 20 and the illumination device 30. The controller 60 can be connected to a computer.

Using the apparatus 1 according to the invention, it is possible to produce images of a bottom side 51 of the cell culture plate 50. Therefore, a glass panel 17 is incorporated in the separation plate 11.

FIG. 2 shows the illumination device 30 from above, said illumination device having a first light source 31, a second light source 32, a third light source 33 and a fourth light source 34. The first light source 31 comprises a plurality of light-emitting diodes (not illustrated), which are arranged next to one another in a row and which emit a strip-shaped light beam 35 over virtually the entire length of the first light source 31. The light beam is illustrated by the parallel arrows 35. Over virtually the entire length thereof, the remaining light sources 32, 33, 34 also each emit a strip-shaped light beam in the direction of the cell culture plate 50. However, corresponding arrows for labelling the respective light beams are not illustrated in FIG. 2 for reasons of clarity.

In addition to the bottom side 51 already mentioned above, the cell culture plate 50 has a top side 52 and a first side face 53, a second side face 54, a third side face 55 and a fourth side face 56. On account of the rectangular basic form of the cell culture plate 50, the opposing side faces 53, 54 extend perpendicular to the other pair of side faces 55, 56 extending in parallel.

The cell culture plate 50 has a multiplicity of wells 57, which are arranged in 8 rows with in each case 12 wells. As can be gathered from FIG. 3, each individual well 57 has a U-shaped form in the longitudinal section. Here, each well 57 can be filled with an aqueous solution from above, i.e. from the top side 52, the dirofilaria and an active ingredient being situated in said aqueous solution. Once filling is complete, the wells can be covered by way of a cover film.

As becomes clear from the overview of FIGS. 2 and 3, the individual light sources 31, 32, 33, 34 each emit light into the cell culture plate 50 through the side faces 53, 54, 55, 56. Thus, light from the first light source passes into the cell culture plate 50 from the outside through the first side face 53. The same applies analogously to light sources 32, 33, 34. In order to achieve the best possible illumination of the individual wells 57 of the cell culture plate 50, the position of the light source 31 can be modified in relation to the cell culture plate 50.

The double-headed arrow 36 indicates that a distance between the first light source 31 and the first side face 53 of the cell culture plate 50 can be increased or reduced. The double-headed arrow 37 (see FIG. 3) indicates that the first light source 31 can be moved upwards or downwards in relation to the cell culture plate 50. Moreover, the first light source 31 can be tilted about a longitudinal axis 38, which extends parallel to the first side face 53 of the cell culture plate 50. The corresponding adjustability about the longitudinal axis 38 or about an axis extending parallel thereto is indicated by the bent arrow 39. Moreover, the light intensity of the first light source 31 can be set by way of the controller 60. The horizontal distance from the respective side face, the vertical position, the inclination angle and the light intensity of the respective light source can be set individually by the corresponding setting parameters of the remaining light sources 32, 33, 34. Consequently, it is possible, for example, to adjust the illumination device in such a way that the light intensity of the first light source 31 deviates not only from the light intensities of the light sources 33, 34 arranged transversely thereto, but also from the light intensity of the opposing light source 32.

The holder 40 has a first receiving corner 41, a second receiving corner 42, a third receiving corner 43 and a fourth receiving corner 44. Preferably, three of the four receiving corners 41, 42, 43, 44 are embodied as stationary stops while the remaining fourth receiving corner is embodied as a sprung stop. A distance between the first receiving corner and the second receiving corner, for example, is dimensioned in such a way that the light beam 35 of the first light source 31 can strike unhindered the region of the first side face in which the wells 57 are situated. Expressed differently, a distance between the receiving corners 41, 42 is greater than a length of the first column of wells, which extends along the first side face 53.

On account of the different lengths of the longer side faces 55, 56 and the shorter side faces 53, 54, the light sources 33, 34 on the one hand and the light sources 31, 32 on the other hand have different lengths. Here, the width of a light beam from a light source is always greater than the length of the region of the associated side face in which the wells are situated.

All regions of the side faces of the cell culture plate 50 in which the wells 57 are situated are impinged by light from the light sources as a result of the illumination device 30 and the special arrangement of the individual receiving corners of the holder 40. As a result of the individual adjustability of the individual light sources, the illumination device 30 can be adjusted in such a way that an almost optimal illumination of the wells 57 is achieved. A preferred monitoring criterion for the illumination that is as optimal as possible has already been described above. An optimal illumination can be assumed when the well-specific results in a cell culture plate whose individual wells have been filled with the same active ingredient in each case do not deviate from one another in respect of the effectiveness of the same active ingredient in each case or have a standard deviation that approximately corresponds to the estimated standard deviation on account of the biological organisms.

### LIST OF REFERENCE SIGNS

1 Apparatus
10 Housing
11 Top separating plate
12 Middle separating plate
13 Top region
14 Middle region
15 Bottom region
16 Opening
17 Glass plate
20 Camera
21 Lens
22 End
23 Conical-frustum-shaped region
30 Illumination device
31 First light source
32 Second light source
33 Third light source
34 Fourth light source
35 Arrow (light beam)
36 Double-headed arrow
37 Double-headed arrow
38 Arrow
40 Holder
41 First receiving corner
42 Second receiving corner
43 Third receiving corner
44 Fourth receiving corner
50 Cell culture plate
51 Bottom side
52 Top side
53 First side face
54 Second side face
55 Third side face
56 Fourth side face
57 Well
60 Controller

1. Apparatus for recording one or more whole-area images of a cell culture plate with one or more wells, comprising
a holder for receiving the cell culture plate with wells, wherein the cell culture plate has a bottom side, a top side and a plurality of side faces, which extend between the bottom side and top side,
a camera that serves to record images of the bottom side of the cell culture plate within a predefined depth of field,
an illumination device for illuminating the cell culture plate, wherein the illumination device comprises a plurality of light sources for producing light beams wherein, in a use position of the cell culture plate,
light of a first light source enters the cell culture plate from the outside through the entire length of a first side face,
light of a second light source enters the cell culture plate from the outside through the entire length of a second side face,
light of a third light source enters the cell culture plate from the outside through the entire length of a third side face, and
light of a fourth light source enters the cell culture plate from the outside through the entire length of a fourth side face,
wherein the intensity of the produced light beams, the vertical height of the produced light beams in relation to the cell culture plate, the horizontal distance of the light sources from the respective side face and the inclination angle of the produced light beams about a longitudinal axis are individually adjustable for each light source by way of means for adjusting the illumination device.

2. Apparatus according to claim 1, wherein the first light source has a Fresnel lens arrangement in order to produce a uniform light over an effective length of the first light source.

3. Apparatus according to claim 1, wherein each light source and/or a holder for fastening the light source is connected to one or more controllers for adjusting the illumination device.

4. Apparatus according to claim 1, wherein the controller for adjusting the illumination device is configured to carry out a method for adjusting the illumination device.

5. Apparatus according to claim 1, wherein the first light source is arranged laterally next to the first side face.

6. Apparatus according to claim 1, wherein the intensity of the produced light beams, the vertical height of the produced light beams in relation to the cell culture plate, the horizontal distance of the light sources from the respective side face and the inclination angle of the produced light beams about a longitudinal axis are set for each light source in such a way that the cell culture plate is illuminated from the bottom to a predefined depth of field.

7. Apparatus according to claim 1, wherein a vertical height of a light beam emerging from the first light source is 2 to 6 mm.

8. Apparatus according to claim 1, wherein the holder has a plurality of receiving corners, wherein a first receiving corner serves to receive one end of the first side face and a second receiving corner serves to receive an opposite end of the first side face, and wherein a distance between the first receiving corner and the second receiving corner is greater than a length of a row or column of wells, which extends along the first side face.

9. Apparatus according to claim 1, wherein the camera comprises a telecentric lens.

10. Apparatus according to claim 1, wherein the camera comprises an image sensor, and the cell culture plate and/or the image sensor are adjustable in relation to one another in all directions such that the cell culture plate is adjusted in all directions parallel to the image sensor.

11. Apparatus according to claim 1, wherein, for purposes of establishing an effect of one or more active ingredients on nematodes and other organisms in aqueous tests, the wells of the cell culture plate are filled with one or more nematodes and/or other organisms in aqueous solutions with and without active ingredients.

12. Method for adjusting the illumination device of the apparatus according to any one of claim 1, wherein, in relation to the cell culture plate, the relative height of each light source, the inclination angle of each light source and a light intensity of each light source are set individually in order to obtain a good illumination of the individual wells of the cell culture plate and a uniform illumination of the cell culture plate.

13. Method according to claim 12, wherein the camera is used to create an image of a monochrome sheet that is situated in a plane above the illuminated cell culture plate and in that the adjustment is implemented on the basis of an evaluation of the image.

14. Method according to claim 13, wherein a histogram of the light intensity is produced from the image and adjustment by way of controlling the means for adjusting the illumination device is implemented on the basis of said histogram until the histogram has a minimum width.
